(19) United States
(12) Patent Application Publication
(10) Pub. No.: US 2023/0044094 A1
(43) Pub. Date: Feb. 9, 2023

(54) METHOD AND SYSTEM FOR IN VITRO SENSING OF ANALYTES

(71) Applicant: Profusa, Inc., Emeryville, CA (US)

(72) Inventors: Ben HWANG, Lafayette, CA (US); Soya GAMSEY, San Francisco, CA (US)

(73) Assignee: Profusa, Inc., Emeryville, CA (US)

(21) Appl. No.: 17/970,339

(22) Filed: Oct. 20, 2022

Related U.S. Application Data
(63) Continuation of application No. PCT/US2021/028419, filed on Apr. 21, 2021.
(60) Provisional application No. 63/013,216, filed on Apr. 21, 2020.

(51) Int. Cl. A61B 5/145; G01N 21/77; G01N 33/53

(52) U.S. Cl. CPC A61B 5/14546; G01N 21/77; G01N 33/53; G01N 2021/7796

(57) ABSTRACT

Some embodiments described herein relate to a method that includes receiving an optical emission signal from a sensor disposed in a vessel. The vessel can be configured for an in vitro biological process (e.g., a bioreactor), and the emission signal can be received while the sensor is in contact with a biological matrix. The emission signal can be received by a reader that is disposed outside the vessel. At least one of a presence, quantity, or concentration of an analyte can be determined based on the emission signal. Similarly stated, the emission signal emitted by the sensor can be dependent on at least one of a presence, quantity, or concentration of the analyte. In some embodiments, the emission signal can be an optical signal emitted by a sensor in response to the sensor being excited by an excitation optical signal emitted by, for example, the reader.

### CROSS REFERENCE TO RELATED APPLICATIONS

This application is a continuation of International Patent Application No. PCT/US21/28419, filed Apr. 21, 2021, which claims the benefit of and priority to U.S. Provisional Patent Application No. 63/013,216, filed Apr. 21, 2020, the entire disclosure of each of which is hereby incorporated by reference.

### FIELD

Embodiments described herein generally relate to systems and methods suitable for sensing analytes in vitro during biological manufacturing processes.

### BACKGROUND

The biomanufacturing field is rapidly adopting new technologies to increase production output of biologics and related products, such as single use bioprocessing technologies. These technologies are cost-effective, enable the quick turnaround of products, and reduce the need for sterilization and reuse thereby reducing the possibility of contamination. While many of the systems that support bioprocessing have kept up with this shift to single use, there are a very few sensors available to support this paradigm shift.

Bioreactions are currently monitored in several ways, for example, by taking aliquots of the contents of the vessel for analysis or by introducing a probe through a sampling or measuring port. Such sampling methods generally do not provide continuous monitoring.

Additionally, removing sample from the bioreactor or introducing a probe introduces a contamination risk. Existing monitoring techniques are generally unsuitable to continuously monitor certain biochemical analytes within a bioreactor while a biological process (e.g., biomanufacturing reaction such as cell culturing) is carried out. For example, while parameters like temperature may be continuously monitored, it is currently not feasible to continuously monitor for the presence, quantity, or concentration certain biochemical analytes without taking aliquots from the vessel or otherwise diverting the contents of the bioreactor.

Accordingly, there is a need for improved sensors, methods of sensing analytes, and systems for sensing analytes that are meet biomanufacturing needs.

### SUMMARY

Disclosed herein are methods and systems for in vitro sensing.

Some embodiments described herein relate to a method that includes receiving an optical emission signal from a sensor disposed in a vessel. The vessel can be configured for an in vitro biological process (e.g., a bioreactor), and the emission signal can be received while the sensor is in contact with a biological matrix. The emission signal can be received by a reader that is disposed outside the vessel. At least one of a presence, quantity, or concentration of an analyte can be determined based on the emission signal. Similarly stated, the emission signal emitted by the sensor can be dependent on at least one of a presence, quantity, or concentration of the analyte. In some embodiments, the emission signal can be an optical signal emitted by a sensor in response to the sensor being excited by an excitation optical signal emitted by, for example, the reader.

Some embodiments described herein relate to a method that includes positioning a reader outside a vessel that contains a biological matrix such that the reader is in optical communication with a sensor disposed within the vessel. The reader can emit an optical excitation signal to illuminate the sensor, for example, through a transparent wall or a fiber optic line. In response to the sensor being illuminated/excited, the sensor can emit and the reader can receive an optical emission signal. The optical emission signal can be dependent on at least one of a presence, a concentration, or a quantity of an analyte in a biological matrix undergoing an in vitro biological process in the vessel. The at least one of the presence, concentration, or quantity of the analyte can be determined based on the optical emission signal while the in vitro biological process is occurring.

Some embodiments described relate to an apparatus that includes a vessel and a sensor. The vessel can be, for example, a bioreactor configured to be used in a biomanufacturing process. Similarly stated, the vessel can be configured such that an in vitro biological process can be performed within. A sensor can be disposed within the vessel. The sensor can be configured to be in contact with a biological matrix while the in vitro biological process occurs and emit an optical emission signal that is dependent upon at least one of a presence, concentration, or quantity of an analyte in a biological matrix. Similarly stated, the sensor can be configured to produce a signal that is capable of allowing for continuous in situ monitoring of an analyte during an in vitro biological process.

In an embodiment, a method of in vitro sensing includes one or more sensors each producing an emission signal in the presence of an analyte. For example, the sensor can be placed into contact with a liquid in vitro. A reader can detect the emission signal. The presence, intensity, spectrum, and/or termporal characteristics of the emission signal can indicate the presence, concentration, and/or quantity of an analyte in the liquid. In an aspect, a reader can send an excitation signal to the sensor such that the excitation signal illuminates and excites a sensing moiety of the sensor. The sensor/sensing moiety can produce an emission signal in response to being excited in the presence of an analyte. The in vitro liquid may include cell culture media. In an aspect, the sensor may continuously analyze for the presence of one or more analytes. The sensor measurement may detect analytes with an accuracy of +/−0.5% accuracy or less. The analyte may be selected from the group consisting of: oxygen, glucose, carbon dioxide, lactate, protons (H⁺), and bicarbonate (HCO₃⁻). The analyte measurements may be used to calculate pH or CO₂. The one or more sensors may detect more than one analyte concurrently. In an aspect, the one or more sensors are located on the surface of a substrate and/or may be located in a sensing container.

A further embodiment relates to a system for in vitro sensing including one or more sensors each operable to produce an emission signal in the presence of an analyte. The sensor(s) can be disposed within a vessel that houses (or is configured to house) a liquid containing biological products and/or components undergoing a biological process. A reader disposed outside the vessel (e.g., on an exterior surface of the vessel and not in contact with the liquid) can be operable to detect the emission signal(s). The one or more sensors may be located on the surface of a substrate. The one or more sensors may be located in a sensing container. The one or more sensors may be physically separate from the device.

An embodiment relates to a method of in vitro sensing, including one or more sensors each operable to produce an emission signal in the presence of an analyte. Each of the one or more sensors can be disposed in one or more of the plurality of vessels. The one or more sensors can be brought into contact with the contents of the plurality of vessels (e.g., the vessels can be filled with biological matrices such as inoculated cell culture media). A plurality of readers can be disposed on the exterior surfaces of the plurality of vessels (e.g., at least one reader can be disposed on each of the plurality of vessels), and each reader can detect the emission signals from sensors in that vessel, such that data from the one or more sensors in the one or more of the plurality of vessels can be analyzed simultaneously. A single analyte may be detected in each vessel, multiple analytes may be detected in each vessel, and/or different analytes can be detected in different vessels. The sensor measurement may detect analytes with an accuracy of +/−0.5% accuracy or less.

An embodiment relates to a system for in vitro sensing including one or more sensors, a vessel, and a reader. Each of the one or more sensor can be operable to produce an emission signal in the presence of an analyte. The vessel can house or configured to house a liquid and the one or more sensors. The reader can be operable to detect the emission signal(s). The reader can be adhered to the exterior surface of the vessel and operable to continuously detects the emission signal(s). The reader can wirelessly (e.g., optically) detect the emission signal. In an aspect, reader may transmit a signal associated with the emission signal data to a hub device. The reader may be flexible such that it can conform to a shape of the surface of the vessel.

### BRIEF DESCRIPTION OF THE DRAWINGS

FIG. 1 illustrates an embodiment of the systems described herein.

FIG. 2 illustrates an embodiment of systems described herein. showing a sensor is placed on an optical fiber such that the reader is detects the signal through the optical fiber.

FIG. 3 illustrates an embodiment of the multiplexing system described herein.

### DETAILED DESCRIPTION

Described herein are methods and systems suitable for in vitro detection and measurement of analytes. These methods and systems are particularly suitable for biomanufacturing and are generally operable to perform aseptic, real-time, continuous monitoring of one or more biochemical analytes. The methods and systems described herein can provide continuous, accurate, and/or automated simultaneous sensing of multiple analytes, without requiring manual sampling or manipulation of the contents of the vessel. The methods and systems described herein can allow continuous analysis throughout a biomanufacturing (e.g., cell culture) process. Once the sensors and reader devices are installed, additional manipulation is not needed for continuous monitoring and/or analysis of the contents of the vessel, for example throughout the duration of an in vitro biological process or bioreaction.

The methods and systems described herein may, in an embodiment, include multiplexing to analyze in the contents of multiple vessels simultaneously.

The methods and systems may include wireless detection of sensors, and wireless transmission of the sensor data.

### Continuous Sensing in Biomanufacturing

There is a need for the ability to continuously, or automatically, detect and measure analytes in biomanufacturing settings. Various techniques exist for monitoring biomanufacturing processes, including removing aliquot or various spectrographic techniques. Spectrographic techniques, however, typically require complex and expensive equipment, may be unsuitable for measuring certain analytes in situ. Invasive manual sampling presents a risk to aseptic processing, can be vulnerable to inconsistent sample collection and analysis, and is not sustainable at commercial manufacturing scale. Permanent or semi-permanent probes, such as thermocouples or pH probes are generally unsuitable for measuring biochemical analytes and generally require a portion of the probe or leads for the probe to extend out of or through the vessel, creating a contamination risk. Accordingly, to achieve sustainable commercial scale manufacturing of tissue engineered products, there is a critical need for the development of robust non-invasive measurement technologies that enable real time monitoring and analysis of critical in-process parameters, including biochemical analytes. The methods and systems described herein address that need.

Exemplary biomanufacturing settings include those for the manufacture of a biologic product including: a monoclonal antibody, a vaccine, a tissue, various proteins, cytokines, enzymes, fusion proteins, whole cells, and viral and non-viral gene therapies.

Biomanufacturing applications of the methods and systems of in vitro sensing include for single-use systems and for multiple use systems.

The methods and systems described herein provide for continuous sensing of analytes in biomanufacturing settings. The methods and systems may include optical readers. The methods and systems provide, in an embodiment, for analysis of the contents of multiple bioreactors simultaneously. Furthermore, multiple analytes may be analyzed, and the multiple analytes may be analyzed simultaneously.

Continuous wireless (e.g., optical) sensing provides several important features in a biomanufacturing setting, including minimizing operations such as fluid transfer or vessel manipulation, which, in turn, minimizes the potential for contamination, the introduction of other manipulation-based factors, and/or sample loss.

Continuous wireless sensing may be used in an open, partially open, closed, or functionally closed bioprocessing system, for example.

Continuous wireless sensing may occur in situ.

### Systems and Methods for In Vitro Sensing

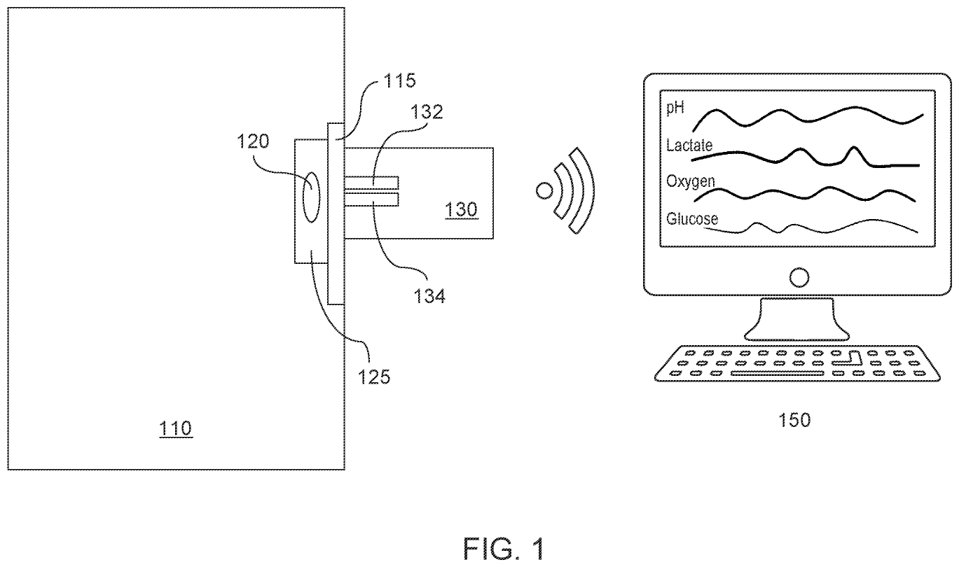

As shown in FIG. 1, systems and methods described herein for in vitro sensing typically includes one or more sensors 120, wherein the one or more sensors produce an emission signal in the presence of an analyte. The sensor 120 can be disposed in a vessel 110 (e.g., a bioreactor) which can contain or be configured to contain a biological matrix. When filled with the biological matrix, the sensor 120 can be in contact with the (usually liquid or gel) biological matrix.

A reader 130 can be operable to detect emission signals from the sensor 120. The reader 130 can be disposed outside the vessel 110 and in optical communication sensor 120. For example, the reader 130 can be adhered to the vessel via a clear adhesive. In some embodiments, no direct physical connection (e.g., wires, fiber optics, etc.) exists between the sensor 120 and the reader 130.

The methods and systems described herein for in vitro biomanufacturing uses were unexpected, especially given the complexities of optical readers and optical sensors.

### Sensors Useful in the Methods of In Vitro Sensing

Examples of sensors useful in the methods and systems described herein described, for example, in U.S. Pat. Nos. 10,117,613; 10,383,557; 10,494,385; and US Patent Application Publication No. 2019/0000364, each of which is hereby incorporated by reference herein in its entirety.

For example, sensors described herein (e.g., sensor 120) can include a polymer scaffold and one or more sensing moieties (also referred to as analyte specific sensing domains) suitable to sense an analyte of interest, including not limited to analyte binding molecules (e.g. glucose binding proteins), competitive binding molecules (e.g. phenylboronic acid based chemistries), analyte specific enzymes (e.g. glucose oxidase, ion sensitive materials, or other analyte sensitive molecules (e.g. oxygen Sensitive des such as porphyrins). The sensing moieties can be incorporated into a scaffold portion by chemical conjugation, physical entrapment, or the like.

The polymer sscaffold can be, for example, a hydrogel. For example, the hydrogel can be prepared by reacting hydroxyethyl methacrylate (HEMA), to form poly(hydroxyethyl methacrylate), pHEMA. Furthermore, various comonomers can be used in combination to alter the hydrophilicity, mechanical and swelling properties of the hydrogel (e.g. PEG, NVP, MAA). Non-limiting examples of polymers include 2-Hydroxyethyl methacrylate, polyacrylamide, N-vinylpyrrolidne, N-Dimethylacrylamide, poly(ethylene glycol) monomethacrylate (of varying molecular weights), diethylene glycol methacrylate, N-(2-hydroxypropyl)methacrylamide, glycerol monomethacrylate, 2,3-dihydroxypropyl methacrylate and combinations thereof. Non-limiting examples of cross-linkers include tetraethylene glycol dimethacrylate, poly(ethylene glycol) (n) diacrylate (of varying molecular weights), ethoxylated trimethylolpropane triacrylate, bisacrylamide and combinations thereof. Non-limiting examples of initiators include Irgacure Series (UV), Azobisisobutyronitrile (AIBN) (thermal), Ammonium Persulfate (APS) (thermal).

An analyte-specific sensing domain may include an analyte detecting portion and an optical signaling portion. The analyte detecting portion and the optical signaling portion may be part of the same molecule or may be different molecules. The analyte detecting portion and the optical signal portion may interact, or be connected chemically, functionally, and the like. Thus, analyte-specific sensing domains can be operable to emit an optical signal in response to an analyte of interest. Analytes of interest described herein are typically biochemical molecules and include, but are not limited to oxygen, glucose, carbon dioxide, lactate, protons (H⁺), and bicarbonate (HCO₃⁻). Such biochemical analytes of interest may be difficult or impossible to detect with known spectrographic techniques and/or other known sensors. Similarly stated, accurately measuring the presence, concentration, and/or quantity of such analytes using known methods may require obtaining samples or the introduction of sensor probes or otherwise running leads into the vessel that risk contamination of the vessel and are generally inoperable to continuously monitor the analytes of interest in real time.

The sensing moieties may be in any form, for example, microspheres, nanospheres, fibers, etc. Non-limiting examples of suitable sensing molecules include but are not limited to dye labeled Concanavalin A with glycodendrimer or dextran (see, e.g., Ballerstedt et al. (1997) Anal. Chin. Acta 345:203-212) and alcohol sensitive oxo-bacteriochlorin derivative fluorescent binding protein developed by Takano, et al (2010) The Analyst 135:2334-2339 as well as Vladimir et al. (2004) Clinical Chemistry 50:2353-2360; Asian et al. (2005) Chem. 1; 77(7):2007-14; Ballerstadt et al. (1997) Anal. Chem. Acta 345:20³-212 (1997); Billingsley et al. (2010) Anal. Chem 82(9):3707-3713; Brasuel et al. (2001) Anal. Chem 73(10):2221-2228; Brasuel, et al. (2003) The Analyst 128(10):1262-1267; Horgan et al. (2006) Biosensors and Bioelectronics 211838-845; Ibey et al. (2005) Anal Chem 77:70390-746; Nielsen et al. (2009) Journal of Diabetes Science and Technology 3(1):98-109: McShane et al. (2000) IEFE Engineering in Medicine and Biology Magazine 19:3645; Mansouri & Schultz (1984) Bio/technology 23:885-890; Rounds, et al. (2007) Journal of Fluorescence 17(1):57-63; Russell et al. (1999) Analytical Chemistry 715):3126-3132; Schultz et al. (1982) Diabetes Care 5:245-253; Srivastava, & McShane (2005) Journal of Microencapsulation 22(4):397-411; Srivastava et al. (2005) Biotechnology and Bioengineering 91(1): 124-131; Takano et al. (2010) The Analyse 135:2334-2339.

In some instances, particularly in instances in which one or more sensing moieties is oxygen-sensitive (e.g., porphyrins), in addition to the sensing moiet(ies) and/or the polymer scaffold, the sensor can include an oxidase, such as, but not limited to, glucose oxidase, and the luminescent dyes (e.g., sensing moieties) and/or their residues incorporated as monomeric units into the polymers measure the consumption of oxygen by the oxidase, thus, the sensors can provide detection of a number of analytes other than oxygen such as, but not limited to, glucose. Other examples of oxidases include bilirubin oxidase, ethanol oxidase, lactate oxidase, pyruvate oxidase, histamine oxidase or other oxidase to provide specificity to other analytes of interest.

As discussed above, one or more sensors 120 can be placed in a vessel 110 containing a biological matrix. In some embodiments, the sensor(s) 120 can be loose in the vessel and configured to sink, float, or be neutrally buoyant. In an embodiment, the sensor may be suspended in the biological matrix. For example, the sensor may be bound to a substrate (e.g., including a fiber optic line) and suspended in a biological matrix. The sensor may be housed in a blister pack.

In other embodiments, however, it may be desirable for the sensor 120 to be fixed to an interior surface of the vessel and/or be bound to a substrate and may be located on the surface of a vessel. For example, as shown in FIG. 1, the sensor 120 can be disposed in a crevice or pocket 125 formed or adhered to an interior surface of the vessel. The pocket 125 can be, for example, a mesh, a fabric mesh, or a polymer mesh. Such a pocket is typically perforated or porous to allow the sensor 120 to be in fluid communication with the bulk contents of the vessel 110. In addition or alternatively, the sensor 120 and/or the pocket 125 can be affixed to a surface of the vessel, for example, using a clear adhesive, through a polymer weld. or using any suitable technique. In an embodiment, sensors may be located on a substrate. The sensors may be located on a substrate using a variety of attachment means, including, but not limited to chemical bonding or physical attachment (including thermal attachment), and the like. Other attachment means known to one of ordinary skill in the art are hereby contemplated herein. The substrate can be a fiber, an optical fiber, the surface of a vessel, a glass surface, a plastic surface, and/or other substrates contemplated by one of ordinary skill in the art.

Typically, the sensor(s) 120 will be fixed to a transparent portion of the vessel 110 such that optical signals can be transmitted to and from the sensor(s) 120. Typically, the sensors 120 are fixed to the vessel 110 in such a manner that the sensor 120 will be directly exposed to the contents of the vessel 110.

In some embodiments, the sensor(s) 120 can be installed during construction of the vessel 110. In other embodiments, sensor(s) 120 can be added by an end user who intends to carry out and/or monitor an in vitro biological process. In such an instance, the end user (e.g., biomedical engineer) can select suitable sensor(s) 120 to monitor a particular biological process. Sensors 120 described herein can be dried, sterilized, and/or otherwise cleaned and remain operable to detect analytes when the vessel 110 is used to contain a biological matrix undergoing a biological process. For example, sensors 120 having a hydrogel scaffold can dehydrate during sterilization and/or storage. Such sensors 120 can rehydrate and be operable to sense analytes when the vessel 110 is filled with a (e.g., gel or liquid) biological matrix. The one or more sensors 120 may be sterilized prior to placement in a vessel. The one or more sensors 120 may be sterilized after placement in a vessel.

In some embodiments, each sensor 120 can be operable to detect a single analyte. For example, each sensor 120 can include an analyte-specific sensing molecule. In such an embodiment, suitable single-analyte sensors can be selected such that each analyte of interest can be detected by one or more sensors 120 configured to emit a signal in dependence upon that particular analyte of interest. In other embodiments, one or more sensors 120 can be operable to detect more than one analyte. Additionally, in some embodiments, sensors 120 can be operable to emit a reference signal that is independent of any analyte. Determining the presence, quantity, and/or concentration of an analyte can include comparing an analyte dependent signal to an analyte independent (or reference) signal.

In an embodiment, the one or more sensors 120 emit and/or transmit an optical signal in response to the presence of an analyte. The optical signal transmitted by the one or more sensors in a vessel may be detected by a reader 130 (also termed a device or a reader device). In an aspect, the reader 130 may be located on the exterior of the vessel 110.

In an embodiment, sensors 120 detect analytes with a % accuracy of +/−5% or less accuracy; +/−0.5% or less accuracy; or +/−0.05% or less accuracy, wherein accuracy refers to what your device is reading relative to the actual true concentration.

### Readers Useful in the Methods of In Vitro Sensing

Suitable readers 130 include, but are not limited to, those described in U.S. Pat. Nos. 10,117,613; 10,045,722; 10,219,729; and US Published Patent Application No. 2016/0374556, which are hereby incorporated by reference herein in their entireties.

A reader typically includes one or more emitter 132, such as a light emitting diode (LED), laser, or other light source configured to emit an optical signal to illuminate and/or excite a luminescent dye or other suitable portion of the analyte-specific sensing domain of the sensor(s) 120. The emitter 132 may also be operable to illuminate and/or excite a reference moiety of the sensor(s). The emitter 132 may be configured to emit light at one or more particular wavelength that correspond to the excitation band(s) of the sensor(s) 120 sensing moieties and/or reference moieties. A reader 130 also typically includes one or more detectors 134, such as a photodiode, charge coupled device (CCD), photomultiplier (SiPM), or other suitable device configured to detect emission signals from the luminescent dye or other suitable portion of the analyte-specific sensing domain. A detector 134 may also be operable to receive signals from reference moieties of the sensor(s).

As the emitter 132 and detector 134 are both optical in nature, the reader 130 can be operable to receive signals from sensors 120 within the vessel 110 in a wireless manner and without drawing samples. For example, reader 120 can be placed on an exterior of a vessel 110 that includes a biological matrix and one or more sensors 120. The reader 120 can be temporarily placed on the exterior of the vessel 110 for a short period of time to obtain an instantaneous (e.g., less than 10 second) measurement of the analyte(s) of interest, or the reader 120 can be mounted to the exterior of the vessel 110 while a bioreaction occurs, such that the analyte(s) of interest can be continuously monitored throughout all or a portion of the biological process.

The reader may transmit data to a data hub. Analysis of the data may occur in the data hub 150. The data hub 150 can be a compute device having a processor and a memory. The data hub can be, for example, a desktop computer, laptop, tablet, server, cloud computing service, or any other suitable computing entity. The data hub 150 can be local, on-site, and/or remote. For example, the data hub 150 can be in the same room or building as sensors 120, readers 130, and/or vessels 110. As another example, the data hub 150 can be at a remote data center or a distributed remote computing environment. The reader 130 can transmit data to the data hub via any suitable wired or wireless technology.

In an embodiment, the reader 130 may be flexible. The flexible reader 130 may conform to the shape of the exterior of a vessel 110. Advantages of this flexibility include optimizing optical performance, including rejection of ambient light, efficient excitation of the sensor, and efficient collection of emission light from the sensor.

### Data Analysis Useful in the Methods of In Vitro Sensing

Data analysis approaches useful in the methods and systems described herein are provided. Exemplary readers include, but are not limited to, those described in U.S. Pat. Nos. 10,117,613; 10,045,722; 10,219,729; US Published Patent Application No. 2016/0374556, and US Published Patent Application No. 2019/0200865, which are hereby incorporated by reference herein in their entireties.

### Multiplexing and Wireless Sensing

There is a need for improvements in the ability to detect and measure analytes in vitro. In particular, improvements are needed in the analysis of multiple bioreactors simultaneously.

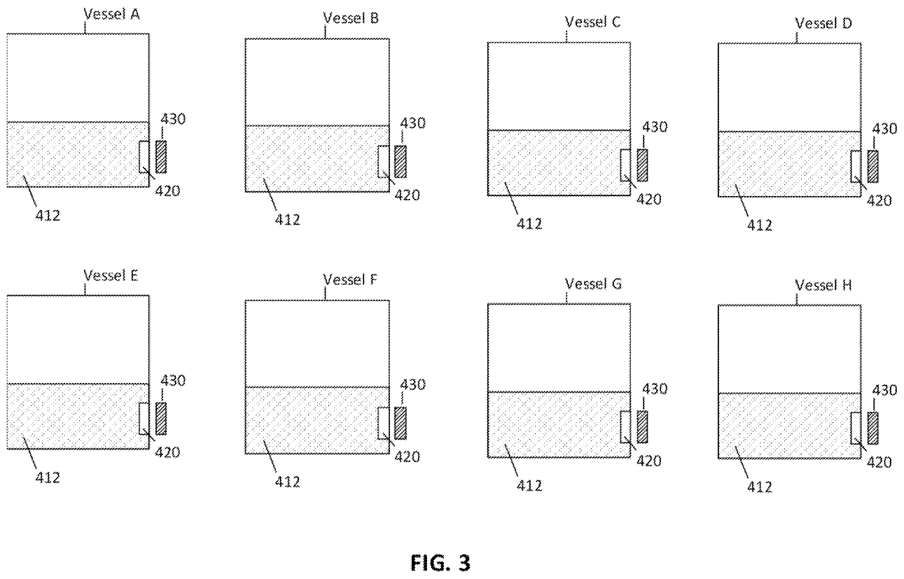

In an embodiment, the method of in vitro sensing may include multiplexing, for example, as shown in FIG. 3. In an aspect, the contents of a plurality of vessels or bioreactors (e.g., vessels A-H shown in FIG. 3) may be analyzed. Each vessel can contain a biological matrix 412. The biological matrix 412 in each vessel can be the same, similar, or different. One or more sensors 420 operable to produce an emission signal in the presence of an analyte may be positioned in each of the plurality of vessels. In an aspect, the sensors 420 in each of the plurality of vessels may detect the same analyte. In an aspect, the sensors 420 in each of the plurality of vessels may detect more than one analyte. In an aspect, sensor(s) 420 in an individual vessel may detect one or more analytes. In an aspect, one or more of the vessels can contain a reference sample (e.g., a sample containing a known quantity or concentration of an analyte of interest).

In an embodiment, the signals from the sensors 420 may be detected by a reader(s) 430. The reader(s) 430 may be located on the exterior surface of a vessel, or a plurality of vessels. The reader(s) 430 may detect signals from the sensors 420 continuously, continuously over a specific period of time, or periodically.

In some embodiments, the reader(s) 430 may simultaneously detect the sensor 420 signals from the plurality of vessels. In other embodiments, a single reader 430 may detect the sensor signals from within a single vessel. For example, a single reader 430 (or any number of readers) can be selectively positioned on an exterior of each vessel from the plurality of vessels. In this way, emission signal(s) generated by the sensor(s) 420 in each vessel can be detected by the reader(s) sequentially without requiring a reader 430 for each vessel. In other embodiments, a multitude of readers 430 may simultaneously detect the sensor signals from within a multitude of vessels. For example, a different reader 430 may be positioned on an exterior of each vessel, such that the contents of each vessel can be monitored simultaneously and/or continuously.

The reader(s) 430 may simultaneously transmit sensor signal data to a data hub. The data may be analyzed in the data hub. The data hub may be located within a physical device, such as a computer or an electronic tablet, and the like. Alternatively, the data hub may be located in the “cloud,” on a server, or on one or more data storage devices. In some embodiments, the reader(s) 430 can be operable to analyze the data locally. For example, the reader(s) 430 can include a processor and a memory and be operable to make an onboard determination of a concentration, quantity, or presence of analyte(s) of interest. Such onboard data analysis can be optionally transmitted to the data hub via any suitable wireless or wired connection. Such onboard data analysis can be transmitted to the data hub while an in vitro biological process is occurring and the reader is receiving optical signals from the sensor(s) 420, or such data can be transmitted to the datahub separately from the monitoring process. For example, the reader(s) can be removed from the vessel(s) and plugged in or wirelessly connected to the datahub.

Sensor signal data from a single reader 430 may be analyzed individually or sensor signal data from more than one reader 430 may be analyzed collectively.

### Substrates and Housing Materials

Sensors described herein may be suspended in a solution, placed on a substrate, or a combination thereof.

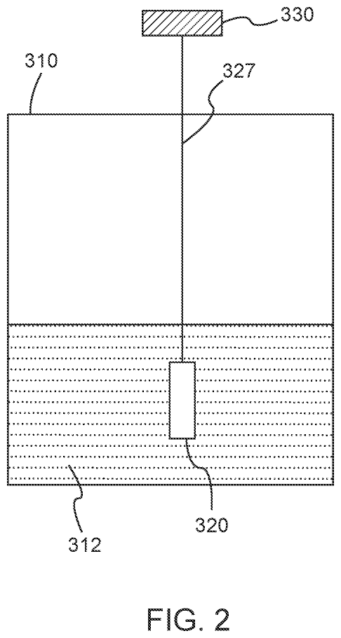

Exemplary substrates include optical fibers, meshes, and the like. For example, as shown in FIG. 2, a sensor 320 can be suspended in a liquid 312. via a fiber optic 327 substrate. In such an embodiment, the sensor 320 can be excited, and the reader 330 can receive emission signals from the sensor 320 via the fiber optic 327 substrate. In addition or alternatively, the substrates and/or the sensor 320 may be placed on the interior surface of a vessel.

### EXAMPLES

### Methods of In Vitro Sensing in Biomanufacturing Applications

Using known methods, a user with a goal of detecting analytes in the production phase of a drug based on mammalian cells to ensure that their batch has a suitable environment for cell growth will have a biomedical engineer manually remove samples from the vessel to measure the analytes of interest that indicate a suitable target environment. The engineer will carry this sample from the production vessel to the Stat Profile Prime Plus system by Nova Bio. The samples will be physically inserted into the device, which then outputs the desired information. At the same time, another engineer wishes to perform the same test (e.g., on a batch in another bioreactor), so they wait until the machine is available for use.

In a prophetic example using methods and systems described herein, tests to measure analytes of interest that indicate a suitable target environment will be performed in vitro. In vitro measurements using methods described herein will allow each of the engineers to test their batches simultaneously at the site of the respective production vessels. Additionally, in vitro methods described herein will prevent potential for contamination because the cell culture fluid (e.g., biological matrix) does not need to be removed in order to run the test. The overall time expenditure between both engineers will be reduced, which helps the company control costs while ensuring quality in their production processes.

### Methods of In Vitro Sensing in Biomanufacturing Applications Using Methods and Wireless Systems Described Herein

In a prophetic example, a small research team desires to perform several cell culture experiments and quantify their results. Their financial and human resources are limited, preventing them from implementing large-scale, capital intensive lab processes and equipment. Using methods and systems described herein, sensors will be placed inside a flask and a wireless reader will be placed outside the flask. They will be able to start with a few flasks and gradually increase their use as their program grows. The in vitro measurements prove to be useful to their scientific process, and they will be able to scale incrementally in accordance with their financial ability.

Methods of In Vitro Sensing in Biomanufacturing Applications Using Methods and Wireless Systems Described Herein to Analyze Multiple Analytes Simultaneously

In a prophetic example, a user in the biopharmaceutical space is searching for a method to address new therapeutic targets. The R&D team identifies a strategy to scan a wide variety of mammalian cell types, each with different suspected optimal growth environments. In order to determine the most effective combination for further production, they will set up an experimental matrix with many small samples. The optimal solution will be determined through a multi-parametric approach. The analysis will include inspecting multi-analyte dynamics within each vessel. Oxygen, glucose, pH, and lactate will be assessed over time in combination with other data points to determine which samples hold the most promise for scale-up. The methods and system described herein will be used to monitor each of these analytes wirelessly in each sample and transmits the data continuously to a central hub. The data science team and engineering team will then collaborate virtually to perform comparative analytics across all samples. From this process, a few candidates will be identified for scale-up and non-viable samples will be quickly discarded, saving the company time and resources.

### Methods of In Vitro Sensing in Biomanufacturing Applications Using Methods and Wireless Systems Described Herein to Analyze a Single Analyte in Multiple Samples Simultaneously

In a prophetic example, a biopharmaceutical company has determined a candidate drug development process for scale-up. They have achieved successful cell growth in small R&D samples and some mid-sized process development batches. Now they will determine the next step is to develop several large production-quantity batches for further testing and validation. In order to proceed to this step, they will ensure strict reproducibility in their processes. They know from experience and research that consistency in oxygenation is a key factor in reproducibility in mammalian cell culture. They will monitor real-time traces of oxygen at each step in the process and use regression analysis to ensure an acceptable level of consistency in oxygen dynamics through each process, irrespective of the scale. Once they have confidence in their ability to produce consistent results at a medium scale, they will proceed to production-level quantities.

### Methods of In Vitro Sensing in Biomanufacturing Applications Using Methods and Wireless Systems Described Herein to Optimize Exchange of Cell Culture Media

In a prophetic example, a biopharmaceutical company has scaled up a certain cell culture process from R&D to the process development phase. At each larger scale, the per batch cost increases significantly. Cell culture media, for example, is a significant variable cost associated with process development. One of the engineering team's goals is to strike the appropriate balance between effective cost management (i.e., not changing cell media too soon) and optimal cell proliferation (i.e., maximizing time in the “log-phase cell growth” stage), which is promoted through the presence of sufficient nutrient availability in the culture. Through use of the methods and system described herein, including multi-analyte detection, the engineers will quantify the boundary conditions within which they will operate before changing cell media. Once the boundary conditions are met, the data analysis hub will trigger an alarm signifying the optimal time to change the cell culture media. Time and cost per batch will be optimized, and data will be available to the team for future process management.

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features and/or combinations of components, other embodiments are possible having a combination of any features and/or components from any of embodiments as discussed above.

Where methods and/or events described above indicate certain events and/or procedures occurring in certain order, the ordering of certain events and/or procedures may be modified. Additionally, certain events and/or procedures may be performed concurrently in a parallel process when possible, as well as performed sequentially as described above.

As used herein, the term “analyte” refers to any molecule or compound to be detected with the methods, apparatus and systems provided herein. Analytes are typically biochemical molecules and/or ions associated with biological processes and/or biochemical reactions, such as cell culturing. Suitable analytes include, but are not limited to, oxygen, glucose, carbon dioxide, lactate, protons (H⁺), and bicarbonate (HCO₃⁻).

What is claimed is:

1. A method, comprising:
receiving, from a sensor, an optical emission signal at a reader while the sensor is disposed in a vessel configured for an in vitro biological process and while the sensor is in contact with a biological matrix within the vessel; and
determining, at the reader disposed outside the vessel, a concentration of an analyte within the biological matrix based on the emission signal.

2. The method of claim 1, further comprising:
placing the sensor in the vessel before the biological matrix is added to the vessel,
the sensor remaining in place while the in vitro biological process occurs.

3. The method of claim 1, wherein:
the sensor in disposed the vessel before the biological matrix is added to the vessel;
the sensor remains in place while the in vitro biological process occurs; and
the concentration of the analyte is determined while the in vitro biological process is occurring.

4. The method of claim 1, further comprising exciting the sensor with an optical signal, the optical emission signal being emitted from the sensor in response to the sensor being excited.

5. The method of claim 1, wherein the sensor is a first sensor, the vessel is a first vessel configured for a first in vitro biological process, the emission signal is a first emission signal, and the concentration of the analyte is a first concentration of a first analyte, the method further comprising:
positioning the reader at the first vessel such that the first emission signal is received;
moving the reader to a second vessel configured for a second in vitro biological process and containing a second sensor;
receiving, from the second sensor, a second emission signal; and
determining at the reader, a second concentration of a second analyte.

8. The method of claim 1, wherein the sensor is from a plurality of sensors, the optical emission signal is from a plurality of optical emission signals, the vessel is from a plurality of vessels configured for a plurality of biological processes, and the reader is from a plurality of readers, the method further comprising:
receiving, simultaneously at each reader from the plurality of readers, an optical emission signal from a sensor from the plurality of sensors, each sensor from the plurality of sensors disposed in a different vessel from the plurality of vessels.

9. The method of claim 8, wherein at least one reader from the plurality of readers receives a first optical emission signal from a first sensor disposed in a first vessel from the plurality of vessels and a second optical emission signal from a second sensor disposed in the first vessel.

10. The method of claim 1, wherein a fiber optic cable optically couples the sensor to the reader.

11. The method of claim 1, wherein the reader receives the emission signal through at least one of a wall of the vessel or an opening of the vessel.

12. A method, comprising:
positioning a reader outside a vessel containing a biological matrix such that the reader is in optical communication with a sensor disposed within the vessel;
emitting, from the reader, an optical excitation signal to illuminate the sensor;
receiving, at the reader and in response to the optical excitation signal, an optical emission signal; and
determining, based on the optical emission signal, a concentration of an analyte within the biological matrix while an in vitro biological process occurs within the vessel.

13. The method of claim 12 further comprising:
placing the sensor in the vessel; and
sterilizing the vessel and the sensor after placing the sensor in the vessel.

14. The method of claim 12, wherein the sensor is a sterile sensor, the method further comprising:
placing the sterile sensor in the vessel.

15. The method of claim 12, further comprising sending a signal associated with the optical emission signal from the reader to a data hub, the concentration of the analyte determined at the data hub.

16. The method of claim 12, wherein the concentration of the analyte is determined at the reader.

17. The method of claim 12, wherein the concentration of the analyte is determined at the reader, the method further comprising:
sending a signal associated with the concentration of the analyte to a data hub.

18. An apparatus, comprising:
a vessel configured for an in vitro biological process; and
a sensor disposed within the vessel, the sensor configured to emit an optical emission signal that is dependent on a concentration of an analyte in a biological matrix undergoing the in vitro biological process.

19. The apparatus of claim 18, wherein the sensor is bound to a wall of the vessel that is transparent to the optical emission signal.

20. The apparatus of claim 18, further comprising an optical fiber coupled to the sensor that is configured to transmit the optical emission signal to an exterior of the vessel.

21. The apparatus of claim 18, further comprising the biological matrix disposed in the vessel.

22. The apparatus of claim 18, wherein the vessel and the sensor are sterilized as a unit.

23. The apparatus of claim 18, wherein the sensor is sterilized before being disposed within the vessel.
